(19) United States
(12) Patent Application Publication
(10) Pub. No.: US 2005/0221382 A1
(43) Pub. Date: Oct. 6, 2005

(54) Stratification of patient populations having or suspected of having rheumatoid arthritis

(76) Inventor: Russell P. Rother, Prospect, CT (US)

Correspondence Address: CDFS - 1087, 445 BROAD HOLLOW RD, SUITE 225, MELVILLE, NY, 11747, US

(21) Appl. No.: 10/508,174

(22) PCT Filed: Mar. 17, 2003
(86) PCT No.: PCT/US03/08226

§ 371 Date: May 11, 2005

Related U.S. Application Data
(60) Provisional application No. 60/365,338, filed on Mar. 18, 2002.

(51) Int. Cl. G01N 33/53

(52) U.S. Cl. 435/7.1

(57) ABSTRACT

The present disclosure is directed to screening, diagnosing and treating patients having, or suspected of having, rheumatoid arthritis. Levels of glucose-6-phophate isomerase or antibodies to glucose-6-phophate isomerase are assayed in test subjects or populations to determine susceptibility to, or existence of, an antibody mediated form of rheumatoid arthritis in such test subjects or populations. The results of the assays provide guidelines for therapeutic intervention with complement inhibiting agents.

### BACKGROUND

1. Technical Field

The present disclosure relates to stratifying patients having, or suspected of having rheumatoid arthritis, and selection of therapeutic agents useful in the treatment thereof.

2. Background of Related Art

Rheumatoid arthritis (RA) is a chronic disease which can exhibit a variety of systemic manifestations. This disease has an unknown etiology and characteristically exhibits a persistent inflammatory synovitis which usually involves peripheral joints in a symmetric distribution. Despite the destructive potential of RA, its course can be quite variable. In many patients, a chronic polyarthritis begins insidiously with fatigue, anorexia, generalized weakness and vague musculoskeletal symptoms until the appearance of synovitis becomes apparent. In about 10% of patients, the onset can be more acute with rapid development of polyarthritis. In about one-third of patients, symptoms may initially be confined to one or a few joints. Overall, most patients will experience an intermediate course of the disease. The potential of inflammation to cause cartilage destruction, bone erosions and, ultimately, joint deformities is the most important feature of this disease.

Many drugs have been used to treat rheumatoid arthritis without complete relief of the symptoms. Conventional drugs include non-steroidal anti-inflammatory drugs (NSAIDs, aspirin, ibuprofen), gold salt, penicillamine, TNFα inhibitors, IL-1 receptor antagonists, and steroidal hormones. The steroidal hormones, which are considered to be the most potent and effective, have side effects when taken for long periods. NSAIDs also have side effects such as stomach irritation and can be ulcerigenic. Recently, a compound which blocks the C5 component of the complement cascade (h5G1.1-mAb) has been shown to be an effective therapy in a murine model of RA. This compound is now in human clinical trials for the treatment of RA.

Although the etiologic stimulus for RA has not been identified, established rheumatoid synovitis is characterized by persistent immunologic activity. The presence of rheumatoid factor, which is an autoantibody reactive with IgG, suggests that there may be an underlying immune dysfunction in association with RA. This factor was initially described by Waaler in 1940 and later described by Rose and co-workers in 1948. Rheumatoid factors consist of IgA, IgG and/or IgM immunoglobulin classes. The idiotypic nature of rheumatoid factor autoantibodies has been described and partially characterized. This suggests that there may be a perturbation in the idiotype/anti-idiotype humoral immune network of patients with RA. In addition, the presence of rheumatoid factor can be of prognostic significance because patients with high titers tend to have more severe and progressive disease with extraarticular manifestations. Rheumatoid factor can also be employed to confirm the diagnosis of RA in patients with a clinical presentation of RA and, if at high titer, to designate patients at risk for severe systemic disease.

The examination of established rheumatoid synovitis reveals a characteristic constellation of features which include hyperplasia and hypertrophy of the synovial lining cells, focal or segmental vascular changes and infiltration with mononuclear cells often collected into aggregates or follicles around small blood vessels. The predominant infiltrating cells are T lymphocytes with T4 (helper-inducer) cells predominating over T8 (suppressor-cytotoxic) cells. Evidence of B cell activation can also be found in the inflamed synovium and plasma cells producing immunoglobulin and rheumatoid factor are characteristic features of rheumatoid synovitis. The resultant production of immunoglobulin and rheumatoid factor can lead to the formation of immune complexes with subsequent complement activation and exacerbation of the inflammatory process through the production of anaphylatoxins and chemotactic factors. Indeed, levels of total hemolytic complement, C3 and C4 are markedly diminished in synovial fluid relative to total protein concentration as a result of activation of the classic complement pathway by locally produced immune complexes. All these findings suggest that progression of RA is an immunologically mediated event.

The most widely used immunodiagnostic assay of RA is the so-called Waaler-Rose assay discussed above. This assay is based upon the antibody (rheumatoid factor) to the Fc region of IgG. Rheumatoid factor (RF) is present in about 60% to 70% of those individuals afflicted with RA. However, the test may not always be accurate because it has been found to give false positives or negatives, and it does not assess the response to therapy or predict activation or reactivation of the disease process.

Since there is no unambiguous test distinguishing RA from other acute or chronic inflammatory diseases, differentiating RA from other arthritides, such as systemic lupus erythematosus (SLE), ankylosing spondylitis (AS), polyarticular gout (PAG), or psoriatic arthritis (PsA) is often difficult. Diagnosis of RA is usually made according to American Rheumatism Association (ARA) criteria, i.e.:
(1) morning stiffness;
(2) joint tenderness or pain on motion;
(3) soft-tissue swelling of the joint;
(4) soft-tissue swelling of a second joint (within three months);
(5) soft-tissue swelling of symmetrical joints (excludes distal interphalangeal joint);
(6) subcutaneous nodules;
(7) X-ray changes;
(8) serum positive for rheumatoid factors;
   wherein diagnosis of 3 or 4 of these factors is considered representative of probable RA and diagnosis of 5 or more of the factors is considered representative of definite RA.

Recently, a transgenic mouse model (K/BxNT cell receptor) was found to exhibit spontaneous inflammatory arthritis resembling many of the features of human RA. See, Matsumoto et al., Science, 286, 1732-1735 (1999) and Ji et al. J. Exp. Med., 194 (3) 321-330 (2001). The inflammatory arthritis was traced to the presence of glucose-6-phosphate isomerase as an antigen targeted by T-cells and IgG immunoglobulins. Glucose-6-phosphate isomerase (“GPI”) is a dimeric enzyme that catalyzes the reversible isomerization of glucose-6-phosphate and fructose-6-phosphate. The protein functions in different capacities inside and outside the cell. In the cytoplasm, the gene product is involved in glycolysis and gluconeogenesis, while outside the cell it functions as a neurotrophic factor for spinal and sensory neurons. The enzyme has also been known as glucosephosphate isomerase, phosphohexose isomerase (PHI), phosphoglucose isomerase (PGI), autocrine motility factor (AMF) and neuroleukin (NLK). In addition, it has recently been shown that a majority of humans with RA have elevated levels of anti-GPI IgG immunoglobulin in sera and elevated levels of soluble GPI in the sera and synovial fluid. See, e.g., Schaller et al., Nature Immunology, Vol. 2, No. 6, 746-753 (2001).

In light of the ambiguity and relative subjectivity of existing tests for RA there is a need for diagnostic procedures that allow non-subjective determination of the existence of RA and which provide a concrete indication of a course of therapeutics.

### SUMMARY

In one aspect, a method for determining the eligibility of a patient having rheumatoid arthritis for treatment with a complement inhibiting agent is provided which includes conducting a glucose-6-phosphate isomerase (GPI) assay of the serum or synovial fluid of a patient to determine the level of GPI in the patient's serum or synovial fluid, and comparing the level of GPI in the serum or synovial of the patient to respective baseline GPI serum or synovial fluid levels established by the test results of a rheumatoid arthritis-free population, wherein the GPI serum or synovial fluid assay results of the patient exceeding the numerical range of the rheumatoid arthritis-free population indicate susceptibility of the rheumatoid arthritis to treatment with the complement inhibiting agent. The patient who exceeds the numerical range may then be treated with the complement inhibiting agent. The complement inhibiting agent may be a C5 complement inhibiting antibody or fragment thereof. The C5 complement inhibiting antibody or fragment thereof may be 5G1.1-mAb, h5G1.1-mAb, 5G1.1-scFv or h5G1.1-scFv. The baseline serum GPI levels may encompass a mean concentration of 0.069+/−0.048 U/ml, P<0.0001. The baseline synovial fluid GPI levels may encompass a mean concentration of 0.060+/−0.052 U/ml P<0001. The patient may be a mammal such as a human.

In another aspect, a method for determining the eligibility of a patient having rheumatoid arthritis for treatment with a complement inhibiting agent is provided which includes conducting an assay for glucose-6-phosphate isomerase antibody (anti-GPI) in the serum or synovial fluid of a patient to determine the level of anti-GPI in the patient's serum or synovial fluid, and comparing the level of anti-GPI in the serum or synovial of the patient to respective baseline anti-GPI serum or synovial fluid levels established by the test results of a rheumatoid arthritis-free population, wherein the anti-GPI serum or synovial fluid assay results of the patient exceeding the numerical range of the rheumatoid arthritis-free population indicate susceptibility of the rheumatoid arthritis to treatment with the complement inhibiting agent. The patient who exceeds the numerical range may then be treated with the complement inhibiting agent. The complement inhibiting agent may be a C5 complement inhibiting antibody or fragment thereof. The C5 complement inhibiting antibody or fragment thereof may be 5G1.1-mAb, h5G1.1-mAb, 5G1.1-scFv or h5G1.1-scFv. The baseline serum anti-GPI levels may encompass a mean concentration of (A₄₀₅) 0.059+/−0.037, P<0.0001. The baseline synovial fluid anti-GPI levels may encompass a mean concentration of (A₄₀₅) 0.645+/−0.209 P<0001. The patient may be a mammal such as a human.

In another aspect, a method for screening a population of subjects for antibody mediated rheumatoid arthritis and determining treatment thereof is provided which includes conducting a glucose-6-phophate isomerase (GPI) assay on the serum or synovial fluid of a population of subjects, and identifying subjects having GPI assay results which are statistically significantly greater than (P<0.05) the respective mean GPI serum or synovial fluid assay results of the population by linear regression analysis, wherein the subjects so identified are diagnosed as having rheumatoid arthritis which is susceptible to treatment with a complement inhibiting agent. The subjects so identified may then be treated with the complement inhibiting agent. The complement inhibiting agent may be a C5 complement inhibiting antibody or fragment thereof. The C5 complement inhibiting antibody or fragment thereof may be 5G1.1-mAb, h5G 1.1-mAb, 5G1.1-scFv or h5G1.1-scFv. The patient may be a mammal such as a human.

In another aspect, a method for screening a population of subjects for rheumatoid arthritis and determining treatment thereof is provided which includes conducting an assay for glucose-6-phophate isomerase antibody (anti-GPI) on the serum or synovial fluid of a population of subjects, and identifying subjects having anti-GPI assay results which are statistically significantly greater than (P<0.05) the respective mean anti-GPI serum or synovial fluid assay results of the population by linear regression analysis, wherein the subjects so identified are diagnosed as having rheumatoid arthritis which is susceptible to treatment with a complement inhibiting agent. The subjects so identified may then be treated with the complement inhibiting agent. The complement inhibiting agent may be a C5 complement inhibiting antibody or fragment thereof. The C5 complement inhibiting antibody or fragment thereof may be 5G1.1-mAb, h5G1.1-mAb, 5G1.1-scFv or h5G1.1-scFv. The patient may be a mammal such as a human.

In another aspect, a method for predicting susceptibility of a patient to rheumatoid arthritis and to treatment with a complement inhibiting agent is provided which includes conducting a glucose-6-phosphate isomerase (GPI) assay of the serum or synovial fluid of the patient to determine the level of GPI in the serum or synovial fluid, and comparing the level of GPI in the serum or synovial fluid of the patient to respective baseline GPI serum or synovial fluid levels established by the test results of a rheumatoid arthritis-free population, wherein the GPI serum or synovial fluid assay results of the patient exceeding the numerical range of the rheumatoid arthritis-free population are diagnostic of susceptibility to rheumatoid arthritis in the patient which is further susceptible to treatment with a complement inhibiting agent. The patient who exceeds the numerical range may then be treated with the complement inhibiting agent. The complement inhibiting agent may be a C5 complement inhibiting antibody or fragment thereof. The C5 complement inhibiting antibody or fragment thereof may be 5G1.1-mAb, h5G1.1-mAb, 5G1.1-scFv or h5G1.1-scFv. The patient may be a mammal such as a human.

In another aspect, a method for predicting susceptibility of a patient to rheumatoid arthritis and to treatment with a complement inhibiting agent is provided which includes conducting an assay for glucose-6-phosphate isomerase antibody (anti-GPI) of the serum or synovial fluid of the patient to determine the level of anti-GPI in the serum or synovial fluid, and comparing the level of anti-GPI in the serum or synovial fluid of the patient to respective baseline anti-GPI serum or synovial fluid levels established by the test results of a rheumatoid arthritis-free population, wherein the anti-GPI serum or synovial fluid assay results of the patient exceeding the numerical range of the rheumatoid arthritis-free population are diagnostic of susceptibility to rheumatoid arthritis in the patient which is further susceptible to treatment with a complement inhibiting agent. The complement inhibiting agent may be a C5 complement inhibiting antibody or fragment thereof. The C5 complement inhibiting antibody or fragment thereof may be 5G1.1-mAb, h5G1.1-mAb, 5G1.1-scFv or h5G1.1-scFv. The patient may be a mammal such as a human.

### DETAILED DESCRIPTION OF PREFERRED EMBODIMENTS

The present disclosure provides convenient, reliable methods to screen and treat patients having, or suspected of having, rheumatoid arthritis (RA). Recognition that glucose-6-phosphate isomerase (GPI) is an antigen which triggers an autoimmune response in a significant portion of the population afflicted with RA provides the basis for these methods. Detection of elevated levels of GPI or antibodies to GPI (anti-GPI) in the sera or synovial fluid of such individuals is indicative of antibody mediated complement activation and provides a signpost for therapeutic intervention through complement inhibition. Thus, by checking for elevated levels of serum or synovial GPI or anti-GPI antibodies in a population of subjects, a determination can be made as to whether members of that population will respond to therapeutic agents that inhibit complement.

In one aspect, patients already diagnosed with RA are stratified with respect to susceptibility to treatment with complement inhibitors. By determining the level of GPI or anti-GPI in serum or synovial fluid, a decision can be made by medical practitioners to treat the RA with a complement inhibitor or some other therapeutic approach. For example, a patient with RA who exhibits elevated GPI or anti-GPI levels may be treated with any pharmaceutically acceptable complement inhibitor. Those RA patients who do not exhibit elevated levels of GPI or anti-GPI would be directed to treatments which are not based on complement inhibition.

It is contemplated that any technique known to those skilled in the art can be utilized to determine the level of GPI or anti-GPI in the serum or synovial fluid. While enzyme-linked immunosorbent assay (ELISA) is preferred for detection of GPI or anti-GPI levels, other assays, e.g., enzyme activity assays, fluid phase assays, radioimmunoassay, precipitation, agglutination, direct and indirect immunofluorescence and complement fixation can be used. These assays may employ any protocol such as competitive, inhibition or sandwich.

Once measured, the GPI or anti-GPI concentrations in serum and/or synovial fluid of non-rheumatoid arthritis patients can be calculated to create a baseline GPI test standard. For example, an assay involving spectrophotometric analysis of enzyme activity to determine the level of GPI in the sera of normal individuals provided a human mean concentration baseline standard of 0.069+/−0.048 U/ml. See, Schaller et al., Nature Immunology, Vol. 2, No. 6, 746-753 (2001). An example of a baseline level of GPI in synovial fluid is 0.060+/−0.052 U/ml, P<0.001. Id. An example of baseline levels of anti-GPI in sera and synovial fluid of healthy individuals was determined by ELISA to be (A₄₀₅) 0.059+/−0.037, P<0.001, and (A₄₀₅) 0.645+/−0.209 P<0001, respectively. Id. Baseline levels may otherwise be determined in accordance with established principles and various techniques known to those skilled in the art.

One can then numerically compare the GPI or anti-GPI assay results of a test subject to the respective GPI or anti-GPI assay results of a rheumatoid arthritis-free population to determine whether the GPI or anti-GPI assay results of the test subject exceed the numerical range of the test results of the rheumatoid arthritis-free population. The numerical comparison involves comparing one or more of the above referenced GPI or anti-GPI results from the test subject to determine if one or more of the results is outside of the numerical range of the test results of the rheumatoid arthritis-free population. Optionally, statistical analysis can be performed to determine if the GPI or anti-GPI test results of the test subject exceed about two standard deviations or more above the mean test results of the rheumatoid arthritis-free population. In another option, statistical analysis can be performed to determine if the GPI or anti-GPI test results of the test subject exceed about one standard deviation or more above the mean test results of the rheumatoid arthritis-free population. These findings are used to support a diagnosis in the test subject of a predisposition to a positive response to complement inhibition therapies.

It should be understood by those of ordinary skill in the art that, in one aspect, this method is useful to diagnose a predisposition for the subsequent development of RA in a test subject or population of test subjects so that an early therapeutic regimen of complement inhibiting agents may be instituted. It should also be understood that not all test subjects with elevated level test results will always develop RA (i.e., there may be false positives). The test results from normal (NL) and RA populations may have overlapping ranges, with RA patients generally having the higher numerical range. The strength of the diagnosis relates to the test results obtained as follows. In a case in which the test results of a test subject lies above, that is, exceeds the numerical range of, the test results obtained from a rheumatoid arthritis-free population, it is said that the test subject is likely to develop RA and that serious consideration of therapy involving complement inhibiting agents should be given. However, not all test subjects that will develop RA will have test results exceeding the numerical range of test results from a rheumatoid arthritis-free population. Therefore, one can perform a statistical analysis to determine if a test result of the test subject is more than two standard deviations above the mean of the test results from a rheumatoid arthritis-free population. The test result of the test subject can be greater than two standard deviations or more above the mean and also be within the absolute numerical range of the results from a rheumatoid arthritis-free population. In this case, it is said that the test subject is likely to develop RA and that complement inhibiting agents should be given consideration. In a final case, the test result of a test subject can be more than one standard deviation above the mean of the test results from a rheumatoid arthritis-free population and also be within the absolute numerical range of test results for RA confirmed patients. The diagnosis of a predisposition for RA and complement inhibiting agent therapy based on these results is less clear. In this case, a test subject may develop RA. However, the percentage of individuals that ultimately develop RA will be smaller than those with test results about two standard deviations or more above normal or those exceeding the numerical range of normal (i.e., an increase in the incidence of false positives).

As mentioned above, it is possible to determine the susceptibility of an entire population of subjects to RA and to determine whether complement inhibiting agents should be given prophylactically in accordance with the present disclosure. The method of screening a population of subjects for the presence or absence of a predisposition toward developing RA includes: (1) performing a GPI or anti-GPI assay on a population of subjects; (2) calculating the results of the GPI or anti-GPI assay for the population; and (3) diagnosing in the test subjects the presence or absence of a predisposition toward developing RA by statistical analysis of the GPI or anti-GPI assay results. If there is a predisposition toward developing RA, prophylactic administration of complement inhibiting compounds should be considered.

The GPI or anti-GPI assay is performed on the population to be screened essentially as described above. Of course, the larger the population of subjects screened, the greater the potential accuracy of the diagnosis. Preferably, the population includes at least about 20 subjects. The GPI or anti-GPI test results for the population of subjects are calculated as described above. The test results are then analyzed statistically to identify subjects having GPI or anti-GPI assay results that are significantly greater than the mean GPI or anti-GPI assay results of the entire population of subjects. For example, a linear regression outlier analysis can be performed on the GPI or anti-GPI assay results for the entire population. Those subjects having one or more GPI or anti-GPI assay results that are statistically significantly greater than the mean values for the population (i.e., the test results is an outlier) are diagnosed as having a predisposition toward developing RA. Although the confidence level for the statistical analysis can be as low as p<0.001, typically, a p<0.05 is used. Linear regression outlier analysis is a known statistical analysis that identifies a data point that differs significantly (i.e., at a given p value) from the mean of a pool of data points from which the data point has been selected. The difference between the data point and the mean of the pool of data points being statistically significantly greater than that which is consistent with the inherent variability of the variable which the data point represents. “p value” or “P<” refers to the probability of observing a sample at least as unlikely as the one observed if the null hypothesis is true. For example, a sample result is statistically significant at the 5% level if, and only if, it leads to a rejection of the null hypothesis when the type I error probability alpha (i.e., the significance level) is 0.05.

Once a determination is made that a subject has elevated GPI or anti-GPI levels, a decision can be made regarding therapeutic intervention. The fact that soluble GPI is present in the synovial fluid of inflamed RA joints indicates that anti-GPI IgG forms immune complexes with the soluble GPI, which precipitate on the surface of the synovial lining with subsequent activation of the complement cascade. See Schaller et al., supra. Accordingly, compounds that interfere with the complement cascade can be utilized to treat subjects having GPI associated RA.

The complement cascade has been studied in depth and is fairly well understood. The complement system is an important means by which a host defends itself against infection. The complement cascade system is a component of the immune system that helps provide a natural immunity against invading microbes and is also an effector arm of antibody mediated humoral immunity. Complement is responsible for activating cells and other molecules involved in the inflammatory process as well as being directly related to the destruction of microbial invaders. The activation of complement involves a cascade of proteolytic reactions that lead to the release of inflammatory mediators and result in the assembly of the terminal complement complex. This cascade system has been characterized as containing at least thirty serum and membrane proteins that are activated by antibody-antigen complexes or by the invasion, in a host or experimentally in culture, by a microorganism, or other antigenic molecules.

The complement cascade consists of two major branches, the classical and alternative pathways. Though these pathways are initiated differently, they converge at the step of complement protein C3 activation. The complement cascade can mediate undesirable cellular damage in inflammatory, immune or autoimmune (auto-antibody-mediated) conditions. A series of regulatory proteins are involved in the control of the complement cascade. These proteins are considered part of the complement system and act to block endogenous complement activity at various stages of the complement cascade.

The classical pathway of the complement system is initialized by complement protein C1 binding to antigen bound IgG or IgM. C4 and C2 are then activated in the formation of C3 convertase at which point an amplification takes place that generates literally thousands of C3a and C3b fragments. C3b fragments can bind to complement protein complex C4b2a to form C4b2a C3b which is called C5 convertase and generates thousands of C5a and C5b fragments. C3b can also be used to regenerate C3 convertase which causes a greater amplification of complement activation. One approach to inhibit complement mediated effects is by depleting complement.

Any compounds that bind to or otherwise block the generation and/or activity of any of the human complement components, such as, for example, antibodies specific to a human complement component are useful herein and are referred to as complement inhibitors or complement inhibiting agents. Some complement inhibiting agents include 1) antibodies or antibody fragments directed against complement components C-1, C-2, C-3, C-4, C-5, C-6, C-7, C-8, C-9, Factor D, Factor B, Factor P, MBL, MASP-1, and MASP-2 and 2) naturally occurring or soluble forms of complement inhibitory compounds such as CR1, LEX-CR1, MCP, DAF, CD59, Factor H, cobra venom factor, FUT-175, y bind protein, complestatin, serine protease inhibitors and K76 COOH.

Particularly suitable complement inhibiting agents for use herein are antibodies or fragments thereof that reduce, directly or indirectly, the conversion of complement component C5 into complement components C5a and C5b. One class of useful antibodies are those having at least one antibody-antigen binding site and exhibiting specific binding to human complement component C5, wherein the specific binding is targeted to the alpha chain of human complement component C5. Such an antibody 1) inhibits complement activation in a human body fluid; 2) inhibits the binding of purified human complement component C5 to either human complement component C3 or human complement component C4; and 3) does not specifically bind to the human complement activation product for C5a. Particularly useful complement inhibitors are compounds which reduce the generation of C5a and/or C5b-9 by greater than about 30%. A particularly useful anti-C5 antibody is 5G1.1-mAb. Also particularly useful is a single chain Fv version of 5G1.1-mAb referred to as 5G1.1-scFv. In a preferred embodiment, humanized versions of these antibodies or scFv referred to as h5g1.1-mAb and h5G1.1-scFv are utilized. Methods for the preparation of these antibodies including h5G1.1-mAb and h5G1.1-scFv are described in U.S. patent application Ser. No. 08/487,283 filed Jun. 7, 1995, now U.S. Pat. No. 6,355,245, and “Inhibition of Complement Activity by Humanized Anti-C5 Antibody and Single Chain Fv”, Thomas et al., Molecular Immunology, Vol. 33, No. 17/18, pages 1389-1401, 1996, the disclosures of which are incorporated herein in their entirety by reference. It is contemplated that Fab fragments, Fab′ fragments, F(ab′)₂ fragments, diabodies, and other forms of antibody fragments directed to the C5 component are suitable as well.

It is intended that the methods of the present disclosure are applicable to various animals such as mammals, e.g., humans, dogs, cats, horses, etc., which can all be benefited by screening and treatment herein. The complement inhibitors can be utilized in pharmaceutical compositions by adding an effective amount of one or more complement inhibitors to a suitable pharmaceutically acceptable diluent or carrier. Those skilled in the art are familiar with numerous techniques and formulations utilized to compound pharmaceutical compositions. The pharmaceutical compositions herein may be administered in a number of ways depending upon whether local or systemic treatment is desired and upon the area to be treated. Administration may be topical, (pulmonary, e.g., by inhalation or insufflation of liquids, powders or aerosols, including by nebulizer; intratracheal, intranasal, enteral, epidermal and transdermal), oral, sublingual, buccal or parenteral. Parenteral administration includes intravenous, intraarterial, subcutaneous, intraperitoneal or intramuscular injection or infusion, intramedullary or intracranial, e.g., intrathecal or intraventricular, administration. Direct injection into affected joints is contemplated as well.

Pharmaceutical compositions for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Compositions and formulations for oral administration include powders or granules, suspensions or solutions in water or non-aqueous media, capsules, sachets, troches or tablets. Thickeners, flavoring agents, diluents, emulsifiers, dispersing aids or binders may be desirable. Compositions for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients. Pharmaceutical compositions herein include, but are not limited to, solutions, emulsions, suspensions, foams and liposome-containing formulations. These compositions may be generated from a variety of components that include, but are not limited to, preformed liquids, self-emulsifying solids and self-emulsifying semisolids, according to conventional methods, by one of skill in the art.

The pharmaceutical formulations herein, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. Further details on techniques for formulation and administration of numerous dosage forms may be found in the latest edition of Remington's Pharmaceutical Sciences (Maack Publishing Co., Easton, Pa.). The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents that increase the solubility of the compounds to allow for the preparation of highly concentrated solutions. For topical or nasal administration, penetrants appropriate to the particular barrier to be permeated are used in the formulation. Such penetrants are generally known in the art.

Pharmaceutical compositions suitable for use herein include compositions wherein the complement inhibitors are contained in an effective amount to achieve the intended purpose. The determination of an effective dose is well within the capability of those skilled in the art. For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, which ameliorates, partially or completely, the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for animal or human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner in light of factors related to the subject that require treatment. Dosage and administration are adjusted to provide sufficient levels of the complement inhibitors to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g per kilogram, depending upon the route of administration. For example, anti-C5 monoclonal antibodies may be administered at about 8 mg/kg or at a 600 mg dose. Anti-C5 scFv may be administered at about 2 mg/kg. Guidance as to particular dosages and methods of delivery is provided in the literature and generally available to practitioners in the art.

Although preferred and other embodiments have been described herein, modifications of the embodiments and further embodiments may be envisioned by those skilled in the art without departing from the scope of the invention as defined by the following claims.

1. A method for determining the eligibility of a patient having rheumatoid arthritis for treatment with a complement inhibiting agent comprising:
conducting a glucose-6-phosphate isomerase (GPI) assay of the serum or synovial fluid of the patient to determine the level of GPI in the serum or synovial fluid; and
comparing the level of GPI in the serum or synovial fluid of the patient to respective baseline GPI serum or synovial fluid levels established by the test results of a rheumatoid arthritis-free population, wherein the GPI serum or synovial assay results of the patient exceeding the numerical range of the rheumatoid arthritis-free population indicate susceptibility of the rheumatoid arthritis patient to treatment with the complement inhibiting agent.

2. A method for determining the eligibility of a patient having rheumatoid arthritis for treatment with a complement inhibiting agent according to claim 1 wherein the complement inhibiting agent is selected from the group consisting of CR1, LEX-CR1, MCP, DAF, CD59, Factor H, cobra venom factor, FUT-175, y bind protein, complestatin, K76 COOH, serine protease inhibitor and antibodies and functional antibody fragments directed against complement components C1, C2, C3, C4, C5, C6, C7, C8, C9, Factor D, Factor B, Factor P, MBL, MASP-1, and MASP-2

3. A method for determining the eligibility of a patient having rheumatoid arthritis for treatment with a complement inhibiting agent according to claim 2 wherein the complement inhibiting agent is a C5 complement inhibiting antibody or functional antibody fragment thereof.

4. A method for determining the eligibility of a patient having rheumatoid arthritis for treatment with a complement inhibiting agent according to claim 3 wherein the antibody is 5G1.1-mAb or h5G1.1-mAb.

5. A method for determining the eligibility of a patient having rheumatoid arthritis for treatment with a complement inhibiting agent according to claim 3 wherein the functional antibody fragment is 5G1.1-scFv or hG51.1-scFv

6. A method for determining the eligibility of a patient having rheumatoid arthritis to treatment with a complement inhibiting agent according to claim 1 wherein the baseline serum GPI levels encompass a mean concentration of 0.069+/−0.048 U/ml, P<0.0001 and the baseline synovial fluid GPI levels encompass a mean concentration of 0.060+/−0.052 U/ml P<0001.

7. A method for determining the eligibility of a patient having rheumatoid arthritis for treatment with a complement inhibiting agent according to claim 1 wherein the patient is a mammal.

8. A method for determining the eligibility of a patient having rheumatoid arthritis for treatment with a complement inhibiting agent according to claim 7 wherein the patient is a human.

9. A method for determining the eligibility of a patient having rheumatoid arthritis for treatment with a complement inhibiting agent comprising:
conducting an assay for antibodies to glucose-6-phosphate isomerase (anti-GPI) in the serum or synovial fluid of the patient to determine the level of ant-GPI in the serum or synovial fluid; and
comparing the level of anti-GPI in the serum or synovial fluid of the patient to respective baseline anti-GPI serum or synovial fluid levels established by the test results of a rheumatoid arthritis-free population, wherein the anti-GPI serum or synovial assay results of the patient exceeding the numerical range of the rheumatoid arthritis-free population indicate susceptibility of the rheumatoid arthritis to treatment with the complement inhibiting agent.

10. A method for determining the eligibility of a patient having rheumatoid arthritis for treatment with a complement inhibiting agent according to claim 9 wherein the complement inhibiting agent is selected from the group consisting of CR1, LEX-CR1, MCP, DAF, CD59, Factor H, cobra venom factor, FUT-175, y bind protein, complestatin, K76 COOH, serine protease inhibitor and antibodies and functional antibody fragments directed against complement components C1, C2, C3, C4, C5, C6, C7, C8, C9, Factor D, Factor B, Factor P, MBL, MASP-1, and MASP-2.

11. A method for determining the eligibility of a patient having rheumatoid arthritis for treatment with a complement inhibiting agent according to claim 10 wherein the complement inhibiting agent is a C5 complement inhibiting antibody or functional antibody fragment thereof.

12. A method for determining the eligibility of a patient having rheumatoid arthritis for treatment with a complement inhibiting agent according to claim 11 wherein the antibody is 5G1.1-mAb or h5G1.1-mAb.

13. A method for determining the eligibility of a patient having rheumatoid arthritis for treatment with a complement inhibiting agent according to claim 11 wherein the functional antibody fragment is 5G1.1-scFv or hG51.1-scFv.

14. A method for determining the eligibility of a patient having rheumatoid arthritis to treatment with a complement inhibiting agent according to claim 9 wherein the baseline serum anti-GPI levels encompass a mean concentration of (A₄₀₅) 0.059+/−0.037, P<0.0001 and the baseline synovial fluid anti-GPI levels encompass a mean concentration of (A₄₀₅) 0.645+/−0.209 P<0001.

15. A method for determining the eligibility of a patient having rheumatoid arthritis for treatment with a complement inhibiting agent according to claim 9 wherein the patient is a mammal.

16. A method for determining the eligibility of a patient having rheumatoid arthritis for treatment with a complement inhibiting agent according to claim 15 wherein the patient is a human.

17. A method for screening a population of subjects for the presence or absence of antibody mediated rheumatoid arthritis and determining treatment thereof comprising:
conducting a glucose-6-phophate isomerase (GPI) assay on the serum or synovial fluid of a population of subjects;
identifying subjects having GPI assay results which are statistically significantly greater than (P<0.05) the mean GPI assay results of the population by linear regression analysis, wherein the subjects so identified are diagnosed as having rheumatoid arthritis which is susceptible to treatment with a complement inhibiting agent.

18. A method for screening a population of subjects for the presence or absence of antibody mediated rheumatoid arthritis and determining treatment thereof according to claim 17 further comprising treating the subjects so identified with a complement inhibiting agent.

19. A method for screening a population of subjects for the presence or absence of antibody mediated rheumatoid arthritis and determining treatment thereof according to claim 18 wherein the complement inhibiting agent is selected from the group consisting of CR1, LEX-CR1, MCP, DAF, CD59, Factor H, cobra venom factor, FUT-175, y bind protein, complestatin, K76 COOH, serine protease inhibitor and antibodies and functional antibody fragments directed against complement components C1, C2, C3, C4, C5, C6, C7, C8, C9, Factor D, Factor B, Factor P, MBL, MASP-1, and MASP-2.

20. A method for screening a population of subjects for the presence or absence of antibody mediated rheumatoid arthritis and determining treatment thereof according to claim 19 wherein the complement inhibiting agent is a C5 complement inhibiting antibody or functional antibody fragment thereof.

21. A method for screening a population of subjects for the presence or absence of antibody mediated rheumatoid arthritis and determining treatment thereof according to claim 20 wherein the antibody is 5G1.1 or h5G1.1.

22. A method for screening a population of subjects for the presence or absence of rheumatoid arthritis and determining treatment thereof according to claim 20 wherein the C5 complement inhibiting antibody wherein the antibody fragment is 5G1.1-scFV or h5G1.1-scFv.

23. A method for screening a population of subjects for the presence or absence of rheumatoid arthritis and determining treatment thereof according to claim 17 wherein the subjects are mammals.

24. A method for screening a population of subjects for the presence or absence of rheumatoid arthritis according to claim 23 wherein the subjects are humans.

25. A method for screening a population of subjects for the presence or absence of antibody mediated rheumatoid arthritis and determining treatment thereof comprising:
conducting an assay for antibodies to glucose-6-phophate isomerase (anti-GPI) in the serum or synovial fluid of a population of subjects;
identifying subjects having anti-GPI assay results which are statistically significantly greater than (P<0.05) the mean anti-GPI assay results of the population by linear regression analysis, wherein the subjects so identified are diagnosed as having rheumatoid arthritis which is susceptible to treatment with a complement inhibiting agent.

26. A method for screening a population of subjects for the presence or absence of antibody mediated rheumatoid arthritis and determining treatment thereof according to claim 25 further comprising treating the subjects so identified with a complement inhibiting agent.

27. A method for screening a population of subjects for the presence or absence of antibody mediated rheumatoid arthritis and determining treatment thereof according to claim 26 wherein the complement inhibiting agent is selected from the group consisting of CR1, LEX-CR1, MCP, DAF, CD59, Factor H, cobra venom factor, FUT-175, y bind protein, complestatin, K76 COOH, serine protease inhibitor and antibodies and functional antibody fragments directed against complement components C1, C2, C3, C4, C5, C6, C7, C8, C9, Factor D, Factor B, Factor P, MBL, MASP-1, and MASP-2.

28. A method for screening a population of subjects for the presence or absence of antibody mediated rheumatoid arthritis and determining treatment thereof according to claim 27 wherein the complement inhibiting agent is a C5 complement inhibiting antibody or functional antibody fragment thereof.

29. A method for screening a population of subjects for the presence or absence of antibody mediated rheumatoid arthritis and determining treatment thereof according to claim 28 wherein the antibody is 5G1.1 or h5G1.1.

30. A method for screening a population of subjects for the presence or absence of rheumatoid arthritis and determining treatment thereof according to claim 28 wherein the C5 complement inhibiting antibody wherein the antibody fragment is 5G1.1-scFV or h5G1.1-scFv.

31. A method for screening a population of subjects for the presence or absence of rheumatoid arthritis and determining treatment thereof according to claim 25 wherein the subjects are mammals.

32. A method for screening a population of subjects for the presence or absence of rheumatoid arthritis according to claim 31 wherein the subjects are humans.

33. A method for predicting susceptibility of a patient to antibody mediated rheumatoid arthritis and treatment with a complement inhibiting agent comprising:
conducting a glucose-6-phosphate isomerase (GPI) assay of the serum or synovial fluid of the patient to determine the level of GPI in the serum or synovial fluid; and
comparing the level of GPI in the serum or synovial fluid of the patient to respective baseline GPI serum or synovial fluid levels established by the test results of a rheumatoid arthritis-free population, wherein the GPI serum or synovial fluid assay results of the patient exceeding the numerical range of the rheumatoid arthritis-free population are diagnostic of a predisposition for antibody mediated rheumatoid arthritis in the patient which is susceptible to treatment with a complement inhibiting agent.

34. A method for predicting susceptibility of a patient to antibody mediated rheumatoid arthritis and treatment with a complement inhibiting agent according to claim 33 further comprising treating the patient exceeding the numerical range with a complement inhibiting agent.

35. A method for predicting susceptibility of a patient to antibody mediated rheumatoid arthritis and treatment with a complement inhibiting agent according to claim 33 wherein the complement inhibiting agent is selected from the group consisting of CR1, LEX-CR1, MCP, DAF, CD59, Factor H, cobra venom factor, FUT-175, y bind protein, complestatin, K76 COOH, serine protease inhibitor and antibodies and functional antibody fragments directed against complement components C1, C2, C3, C4, C5, C6, C7, C8, C9, Factor D, Factor B, Factor P, MBL, MASP-1, and MASP-2.

36. A method for predicting susceptibility of a patient to antibody mediated rheumatoid arthritis and treatment with a complement inhibiting agent according to claim 35 wherein the complement inhibiting agent is a C5 complement inhibiting antibody or functional antibody fragment thereof.

37. A method for predicting susceptibility of a patient to antibody mediated rheumatoid arthritis and treatment with a complement inhibiting agent according to claim 36 wherein the wherein the antibody is 5G1.1 or h5G1.1.

38. A method for predicting susceptibility of a patient to antibody mediated rheumatoid arthritis and treatment with a complement inhibiting agent according to claim 36 wherein the antibody fragment is 5G1.1-scFV or h5G10.1-scFv.

39. A method for predicting susceptibility of a patient to antibody mediated rheumatoid arthritis and treatment with a complement inhibiting agent according to claim 33 wherein the patient is a mammal.

40. A method for predicting susceptibility of a patient to rheumatoid arthritis according to claim 39 wherein the patient is a human.

41. A method for predicting susceptibility of a patient to antibody mediated rheumatoid arthritis and treatment with a complement inhibiting agent comprising:
conducting an assay for antibodies to glucose-6-phosphate isomerase (anti-GPI) in the serum or synovial fluid of the patient to determine the level of anti-GPI in the serum or synovial fluid; and
comparing the level of anti-GPI in the serum or synovial fluid of the patient to respective baseline anti-GPI serum or synovial fluid levels established by the test results of a rheumatoid arthritis-free population, wherein the anti-GPI serum or synovial fluid assay results of the patient exceeding the numerical range of the rheumatoid arthritis-free population are diagnostic of a predisposition for antibody mediated rheumatoid arthritis in the patient which is susceptible to treatment with a complement inhibiting agent.

42. A method for predicting susceptibility of a patient to antibody mediated rheumatoid arthritis and treatment with a complement inhibiting agent according to claim 41 further comprising treating the patient exceeding the numerical range with a complement inhibiting agent.

43. A method for predicting susceptibility of a patient to antibody mediated rheumatoid arthritis and treatment with a complement inhibiting agent according to claim 42 wherein the complement inhibiting agent is selected from the group consisting of CR1, LEX-CR1, MCP, DAF, CD59, Factor H, cobra venom factor, FUT-175, y bind protein, complestatin, K76 COOH, serine protease inhibitor and antibodies and functional antibody fragments directed against complement components C1, C2, C3, C4, C5, C6, C7, C8, C9, Factor D, Factor B, Factor P, MBL, MASP-1, and MASP-2.

44. A method for predicting susceptibility of a patient to antibody mediated rheumatoid arthritis and treatment with a complement inhibiting agent according to claim 43 wherein the complement inhibiting agent is a C5 complement inhibiting antibody or functional antibody fragment thereof.

45. A method for predicting susceptibility of a patient to antibody mediated rheumatoid arthritis and treatment with a complement inhibiting agent according to claim 44 wherein the wherein the antibody is 5G1.1 or h5G1.1.

46. A method for predicting susceptibility of a patient to antibody mediated rheumatoid arthritis and treatment with a complement inhibiting agent according to claim 44 wherein the antibody fragment is 5G1.1-scFV or h5G1.1-scFv.

47. A method for predicting susceptibility of a patient to antibody mediated rheumatoid arthritis and treatment with a complement inhibiting agent according to claim 41 wherein the patient is a mammal.

48. A method for predicting susceptibility of a patient to antibody mediated rheumatoid arthritis and treatment with a complement inhibiting agent according to claim 47 wherein the patient is a human.
